(19) United States
(12) Patent Application Publication
(10) Pub. No.: US 2007/0287994 A1
(43) Pub. Date: Dec. 13, 2007

(54) Endoscopically Introducible Expandable Bipolar Probe

(76) Inventor: Pankaj Amrit Patel, Granger, IN (US)

Correspondence Address: PANKAJ A. PATEL, 50726 LAKESIDE DRIVE, GRANGER, IN, 46530, US

(21) Appl. No.: 11/309,026

(22) Filed: Jun. 12, 2006

(51) Int. Cl. A61B 18/18

(52) U.S. Cl. 606/41

(57) ABSTRACT

A device and method for treating tissue inside a patient's body, the device includes an endoscopically introducible catheter shaft. An expandable chamber is mounted on the distal end of the catheter shaft. The chamber is defined by a flexible non-elastomeric wall. The chamber is associated with a first lumen defined by the catheter for fluid flow between the chamber and a fluid source outside of the patient's body. The chamber is filled with fluid after placement in the patient's body. When the expandable chamber is filled with fluid it has a diameter greater than the diameter of the transverse cross-section of the endoscope channel. According to the method, the endoscope is inserted into a patient's body and is used to view the inside of the patient's body, to determine the location of tissue to be treated. The catheter is inserted into the channel that passes through the endoscope. The wall of the expandable chamber is covered with electrodes connectable to an external radiofrequency electrical potential. The chamber is filled with fluid and is positioned at the location of tissue to be treated and the electrical potential is applied to the electrodes resulting in treatment of the area of the body by tissue resistive electrocautery.

### BACKGROUND OF INVENTION

1. Field of Invention

This invention relates to the general field of endoscopic medical devices and specifically to those devices used for ablation of lesions and control of bleeding using bipolar or multipolar cautery technique in the medical field.

2. Prior Art

The use of heat for the cauterization of tissue dates to ancient times. In the present century the use of radio frequency (RF) electrical current traveling through a portion of the body has been widely used to stop bleeding. Cauterization of tissue arises by virtue of its resistance to the passage of RF energy. In the cauterization of bleeding, the proteins in the tissue are heated to a temperature where the proteins congeal and the walls of bleeding vessels are coapted or welded together to stop the bleeding. RF energy is preferred because its frequency is above that which could otherwise cause neuro-muscular stimulation. Several modes of RF cauterization of tissue are employed, such as monopolar or bipolar coagulation.

In monopolar coagulation, an active electrode of small dimensions such as of the order of one to two mm is applied to the bleeding site and the current path is completed through the body to a distal plate electrically in contact with a large surface area of the body such as the buttocks or back. One technique in which the monopolar mode may be employed involves fulguration which is the use of a spark or arc from the active electrode to the tissue. In bipolar coagulation, the two active electrodes are closely spaced, of the order of millimeters so that the current path is confined to a local region of the tissue.

Another technique for stopping bleeding involves the delivery of thermal energy, such as from a resistively heated probe as described in an article entitled “The Heater Probe: A New Endoscopic Method For Stopping Massive Gastrointestinal Bleeding” by David C. Auth et al and appearing in Vol. 74, No. 2, Part 1, pages 257-262 of Gastroentology, 1978. Laser energy has been suggested as described in an article entitled Endoscopic Laser Treatment by David C. Auth et al and appearing at pages 232-239 of the above Gastroentology publication.

A comparison of these various coagulating techniques appears at pages 362-366 of an article entitled “Nonsurgical Management Of Acute Nonvariceal Upper Gastrointestinal Bleeding” by David C. Auth et al and published at page 349 of Hemostasis and Thrombosis, Vol. 4, 1979, Edited by T. H. Spaet, published by Grune & Stratton, Inc. The superiority of bipolar cautery as compared to heater probe or monopolar cautery is described in that publication. When a tissue area is to be treated, each source of blood is subjected to thermal treatment. This means the clearing of tissue with a wash of fluid, followed by the application of heat, again clearing the area and applying heat and so on until all of the bleeding areas have been coagulated. In such treatment, the repeated applications should be made with facility in an accurate manner with a minimum of undesirable side effects such as the sticking of the coagulating device to tissue areas. The laser technique has the advantage of not requiring physical contact, and thus avoiding such sticking problems, but because of the variable way in which different tissue conditions permit absorption of the laser energy, precise control during tissue treatment is difficult. The monopolar electrosurgical device tends to injure tissue not intended to be treated and even cause damage in the target area itself such as by excessively deep effects in the target area. The heater probe tends to stick to the tissue and when the probe is removed following treatment there is often a tearing of the tissues that can precipitate further bleeding. Hence, bipolar electrosurgical treatment of tissue has been used and proposed as improving safety, efficacy and ease of use because the electric current is confined to the small area between the electrodes.

In the medical field, to provide care to patients, there is often a need to ablate lesions that may include dilated blood vessels (vascular malformations), neoplastic lesions (early cancers) or control bleeding from blood vessels that have been eroded and exposed by invading stomach or duodenal ulcers. These lesions are usually located deep within the body and cannot be easily reached except with specialized instruments such as endoscopes, colonoscopes, bronchocopes and cystoscopes.

Typically these specialized instruments (endoscopes, colonoscopes, bronchocopes and cystoscopes) are of thin caliber because they need to be passed via small natural orifices (mouth, rectum, nares, urethra) along the thin internal passageways to the point of interest where the lesion is located. For example, the endoscope that is used to evaluate the upper gastrointestinal tract (UGI tract) measures 9 mm in diameter and is 140 cm in length and can be passed via the mouth to evaluate the UGI tract including the esophagus, stomach and duodenum. Similarly the colonoscope which is used to evaluate the colon measures 12-13 mm in diameter and is 180 cm in length and it can be passed through the rectum and used to evaluate the entire colon and terminal ileum. These specialized instruments typically all have a working channel that runs the length of the instrument to allow the manipulating physician to pass elongated instruments from the exterior along the entire length of the instrument all the way to the tip of the instrument and a little beyond it to obtain biopsies, resect lesions, ablate lesions and cauterize lesions that are located deep within the body.

The working channel of these specialized instruments (endoscopes, colonoscopes, bronchocopes and cystoscopes) are of very small caliber and can usually only accommodate elongated accessories that have a diameter of 3.2 mm or less. Quite often during endoscopy and colonoscopy lesions are encountered that need to be ablated by electrocautery technique. The ablation of these lesions usually requires the use of a bipolar cautery probe that is passed via the working channel of an endoscope into the internal part of the body of the patient to the sight of the lesion. Typically these probes are long (180 cm or more) and of thin caliber 2.2-3.2 mm. All the bipolar cautery probes available are limited in size to 3.2 mm or less because this is the maximum diameter of the working channel of the endoscope and colonoscope. Quite often however the lesions encountered are large blood vessels measuring 5 mm or more in size and require a bipolar cautery probe of larger diameter to effectively, easily and safely ablate the lesion however all the bipolar cautery probes available are limited in size to 3.2 mm because this is the maximum diameter of the working channel of the endoscope and colonoscope. Similarly bleeding vessels seen in the base of eroding gastric or duodenal ulcers are of diameter 4-5 mm and can be very difficult to ablate using the standard 3.2 mm bipolar cautery probe due to the size discrepancy between the instrument and the lesion. The limitation in the size of the tip of the bipolar instrument also increase the time it takes to ablate the lesion and also increases the likelihood of incomplete ablation and subsequent complications. In addition to the limitation in the size of the bipolar cautery probes one of the other disadvantages of the bipolar cautery probes is its cylindrical cross-section and flat tip that limits the ability to achieve close apposition to the tissue to be ablated. Since the interior of the GI tract has a concave configuration when viewed from the lumen, tangential application of the cylindrical bipolar cautery probe often does not provide effective tissue contact and hemostasis.

Several hemostatic thermal type probe devices have been described. For example, starting with an early 1875 U.S. Pat. No. 164,184 to Kidder, a bipolar electrosurgical device is proposed wherein a pair of conductors are spirally wound onto a rubber probe body in which the conductors are embedded. The conductors are shown terminated at a distal hemispherically shaped end of the probe body. A thermally heated knife is described and shown in the U.S. Pat. No. 1,366,756 to R. H. Wappler who employed a pair of half-round cross-sectionally shaped conductor rods twisted about an insulator to connect to a heater-knife. In 1934 Kimble proposed a bipolar electrosurgical device in U.S. Pat. No. 1,983,669 wherein a pair of conductors are shown twisted around a common insulator and project from a retainer body in a manner useful for side-wise or head-on application to a tissue area.

The U.S. Pat. No. 4,011,872 to Komiya proposes an electrosurgical device wherein, for example, as shown in FIGS. 5, 9 and 11, one conductor is connected to a high frequency energy source and is formed of three or four electrodes. The electrodes individually extend from a distal end with spacings between electrodes being variable to accommodate or grasp differently sized tissue areas. In the U.S. Pat. No. 3,987,795 to Morrison, an electrosurgical device is described to operate in a mode which is intermediate between the mono and bipolar modes of electrosurgery. This is achieved by mounting on one body, made of ceramic or glass, an active electrode and a return electrode whose surface area is made significantly larger than that of the active electrode.

The most popular bipolar cautery probe on the market today and routinely used by gastroenterologist to control bleeding from ulcers or vascular malformations is the GoldProbe made by Boston Scientific. It comes in different sizes, but the maximum diameter size available is 3.2 mm to pass through a therapeutic endoscope or colonoscope. It has a cylindrical cross-section. The major limitations of this instrument is that its size is small compared to the size of lesions that need to be routinely treated such as bleeding ulcers or vascular malformations. In addition, the cylindrical cross-section limits the utility in achieving apposition to the tissues that need to be cauterized, especially with tangential application and en-face application to the tissue. Also the small 3.2 mm tip limits the utility when applying the instrument en-face to the tissue, because the small tip reduces the contact area significantly.

Abele et al in U.S. Pat. No. 5,103,804 describes an expandable tip hemostatic probe. The device by Abele et al. includes an endoscopically introducible catheter shaft. A chamber is mounted on the tip of the catheter shaft. The chamber is defined by a flexible wall. The chamber is associated with a first lumen defined by the catheter for fluid flow between the chamber and a fluid source outside of the patient's body. The chamber is fillable with fluid after placement in the patient's body. When the chamber is filled with fluid it has a diameter greater than the diameter of the transverse cross-section of the endoscope channel. According to the method, the endoscope is inserted into a patient's body and is used to view the inside of the patient's body, to determine the location of tissue to be treated. The catheter is inserted into the channel that passes through the endoscope. The chamber is filled with a resistive fluid and is positioned at the location of tissue to be treated. The chamber has a heating device on its inside that maintains the temperature of the inflation liquid at a predetermined elevated tissue-treating temperature. Basically the device is an expandable heater probe. In the endoscopically introducible probe of Abele heating is via a heating device located within the expandable chamber for causing electrical current to flow through the resistive liquid within the chamber to heat the liquid on the basis of l*l*R losses of electrical current flowing through the liquid, and the liquid in turn heating surrounding tissue by thermal conduction through the wall of the expandable chamber. The disadvantage of this device is that the expandable chamber is flexible which means that when inflated it will not form a rigid chamber that can be applied with adequate pressure to ablate an active bleeding vessel. Ablating an actively bleeding vessel usually requires significant pressure n the walls of the bleeding vessel to allow a coapting or welding together of the vessel walls so that the bleeding subsides. With a chamber made of flexible material the flexibility will allow deformation of the expandable chamber and a loss of tamponading pessure to coapt or weld together the walls of the bleeding vessel. To some extent the GoldProbe by Boston Scientifc overcomes the flexibility by using a rigid stiff unbending plastic that does not flex to provide a rigid structure for application of tamponade pressure to weld together the walls of the bleeding vessel. The other disadvantage of the Abele et al device is that it requires the chamber to be filed with a specialized resistive fluid to generate the heat to provide a cautery effect, and it also requires the use of a special external temperature control and RF power supply control unit. These specialized power control units that heat up the tip of the hemostatic probe by heating up a resistive fluid within the expandable chamber are not even available on the market. The device by Abele et al is not a bipolar cautery type device and hence cannot be used with the widely available bipolar cautery units by ERBE (GMBH, Germany), Conmed, Endostat or Valleylab. Due to the above defined limitations the device invented by Abele et al is not commercially available and the power control unit for this type of hemostatic device is also unavailable. Another disadvantage of this device with the heater probe type thermal cautery effect is that it is more prone to stick to the tissues during control of bleeding. Subsequently the tearing of the tissues as the probe is lifted off the tissues often precipitates recurrent bleeding and this often becomes a viscous cycle. Whereby, the heater probe controls bleeding but than sticks to the tissues and when attempts are made to remove the probe, bleeding recurs and the probe needs to be reapplied and so on and so forth.

The invention by Lennox et al described in U.S. Pat. No. 4,955,377 is very similar to the device by Abele et al. It is a device and method for heating tissue, the device having a catheter shaft for insertion into a patient's body, a chamber formed by a expandable balloon mounted on the catheter shaft and filled with an electrically conductive fluid, two or more electrical contacts enclosed within the chamber, a power supply for applying an electrical potential to the contacts, and a two or more conductors for connecting each of the contacts to the power supply. The fluid is heated on the basis of l*l*R losses by a radio frequency electric current flowing between the electrodes, and the fluid in turn heats the surrounding tissue by heat transfer through the wall of the chamber. According to the method, the apparatus is inserted into the patient's body, the chamber is filled with an electrically conductive fluid, and an electrical potential is applied to the contacts. The apparatus functions as a temperature source. A thermister sensor in the balloon or in contact with tissue responds to the heating effect to control the application of the current. Advantageously, by periodic sensing of temperature, and application of controlled rf power, a preset constant temperature is maintained at the selected sensing point, either at the internal body site or the liquid within the balloon. In this way carefully controlled therapy can be conducted at constant temperature. All the limitations that apply to the device by Abele et al are directly applicable to the device by Lennox et al.

U.S. Pat. No. 4,532,924 by Auth et al is a multipolar electrosurgical device described for use in neurosurgery or through the channel of an endoscope or other precision surgery procedures. The device is formed with an insulative probe body, which, in the described embodiment, is sized to pass through a channel of an endoscope to enable the electrocoagulation of blood vessels such as may be needed in the treatment of a gastrointestinal ulcer. The probe body is provided with electrically separate conductors which are formed of a plurality of electrodes distributed over the peripheral surface of the probe body. The electrically separate conductors are so sized in width W and spaced from each other by a distance S as to establish a ratio of W:S which enables effective bipolar electrocautery thermal treatment of tissuel. A plurality of at least six electrodes which can form six bipolar electric fields are formed which in one embodiment are aligned longitudinally on the probe body. The electrodes extend onto the probe body's distal end to provide an omnidirectionally effective electrosurgical device. A central conductive wash channel is provided for electrical connection to a set of electrodes at the distal end of the probe body while also providing a passage for fluid to enhance the visibility of the target area for subsequent precise electrocoagulation of the bleeding site. This particular device is the basis of the GoldProbe by Boston Scientific and is widely used. The limitations of this device are that its size is small compared to the size of lesions that need to be routinely treated such as bleeding ulcers or vascular malformations because the size of the instrument is limited to what can pass through the working channel of an endoscope that is 3.2 mm. In addition, the cylindrical cross-section limits the utility in achieving apposition to the tissues that need to be cauterized, especially with tangential application to the tissue, which is often the case when trying to control internal bleeding from a concave surface of the GI tract. Also the small 3.2 mm tip limits the utility when applying the instrument en-face to the tissue, because the small tip reduces the contact area significantly. Treatment of larger lesions will also take an increased length of time due to the disparity between the size of the catheter 3.2 mm maximum and the size of the lesion to be treated. Another disadvantage of this device is that it is more prone to stick to the tissues during control of bleeding. Subsequently the tearing of the tissues as the probe is lifted off the tissues often precipitates recurrent bleeding and this often becomes a viscous cycle. Whereby, the heater probe controls bleeding but than sticks to the tissues and when attempts are made to remove the probe, bleeding recurs and the probe needs to be reapplied and so on and so forth.

U.S. Pat. No. 4,449,528 by Auth et al describes a miniaturized, endoscopically deliverable thermal cautery probe for cauterizing internal vessels. The probe is applied to tissues cold, and a large number of electric heating pulses of equal energy are then applied to an internal heating element in the probe. The probe has an internal heating element in direct thermal contact with an active heat-transfer portion that has a low heat capacity to insure quick heating and subsequent cooling, thereby adequately coagulating tissue while minimizing heat penetration and resulting tissue damage. The electrical power applied to the probe is continuously measured and is terminated when the energy delivered reaches a preset value. The number of such pulses applied to the probe (and hence the total energy delivered) may be preset while the duration of the period during which the pulses were applied is displayed. Alternatively, the duration of the period during which such pulses are applied to the probe may be preset while the number of pulses applied (and hence the total energy delivered) is displayed. The heating element for the probe is a controlled breakdown diode which has a breakdown voltage that is a function of its temperature so that the temperature can be controlled. The heating element has a resistance of greater than 0.5 ohm to provide adequate power dissipation with relatively low currents. A washing fluid, preferably flowing along the outside of the probe toward its tip, cleans blood from the tissue to be coagulated to make the source of blood more readily visible. The disadvantage of this device is that it is not a bipolar cautery device and hence cannot work with the most commonly used power control units available in GI labs around the world i.e. the machines by ERBE (GBMH), Conmed and Valleylabs. It requires its own specific power and control unit which are not widely available. It also suffers from all the disadvantages of the Goldprobe by Boston scientific which include that its size is small compared to the size of lesions that need to be routinely treated such as bleeding ulcers or vascular malformations because the size of the instrument is limited to what can pass through the working channel of an endoscope that is 3.2 mm. In addition, the cylindrical cross-section limits the utility in achieving apposition to the tissues that need to be cauterized, especially with tangential application to the tissue, which is often the case when trying to control internal bleeding from a concave surface of the GI tract. Also the small 3.2 mm tip limits the utility when applying the instrument en-face to the tissue, because the small tip reduces the contact area significantly. Treatment of larger lesions will also take an increased length of time due to the disparity between the size of the catheter 3.2 mm maximum and the size of the lesion to be treated. The invention by Hiltebrandt described in U.S. Pat. No. 3,920,021 relates to devices for coagulating animal tissue by means of high frequency current. Such devices are known to include two electrodes connectable to sources of high frequency alternating current at different potentials, and the coagulating current flows between these electrodes after they have been applied to the body tissue. Such devices also further consist of a barrel with a coagulator fitting provided at the distal end thereof. In accordance with the invention the coagulator fitting in a device of the kind just described utilizes two electrodes which are separated from one another by an insulator, and these are arranged at the distal end of the barrel. The previously described limitations of small size, poor tissue apposition with en-face and tangential application when trying to control bleeding from concave internal body surfaces also apply to this device.

U.S. Pat. No. 4,709,698 describes an invention by Johnston et al that is a bipolar heatable dilation catheter that is used to dilate strictures. It is for dilating narrowed vessels and malignant or benign obstruction in the GI tract it is not applicable to hemostasis and control of bleeding because of the size and shape and placement of the device. The device by BARRX Medical is a large balloon (22-34 mm) catheter with transversely arranged multipolar electrodes restricted to only the mid-portion of the balloon, it is designed to ablate Barretts esophagus only. There are many disadvantages to this particular catheter. It is too large to pass through the channel of the endoscope, hence cautery can only be applied blindly over a guidewire. The large size allows application of cautery only in the esophagus. It is too large to maneuver to apply cautery in the stomach or duodenum. The shape of the balloon and the restriction of the multipolar electrodes to the midportion of the cylindrical balloon preclude application of cautery in the en-face, tangential or downstream position. Hence this device is only useful to operate to ablate Barretts. In addition this device requires a special RF control module specific to the use of the catheter to ablate Barretts. It delivers less than 1 sec of controlled electrical energy to provide a controlled depth of injury. The duration of energy application is insufficient to control bleeding vessel or ablate blood vessels as it can take 5-10 seonds of treatment to control bleeding. It cannot be used with the generally available bipolar RF control modules such as the ERBE, Conmed, ValleyLab or Endostat.

U.S. Pat. No. 6,238,392 B1 by Gary Long describes a bipolar balloon electrode system whereby the electrodes are mounted on two separate balloon and it is only useful for ablation of Barretts esophagus. It is a large device, long and unwieldy and cannot be used for control of tissue bleeding. It also is sized and shaped for use only in the esophagus and cannot be used in the colon, stomach or duodenum.

U.S. Pat. No. 4,979,948 by Leslie Geddes et al is a balloon based device for ablation of the lining of the gall bladder. It consists of a central electrode and a second electrode on the balloon with transmission of current within the balloon from the central electrode to the conductor on the inner surface of the balloon. This device is shaped to ablate the entire lining of the gall bladder. It is too large to be used in a targeted manner for control of tissue bleeding. It is shaped to fill up the gall bladder which makes it unwieldy to be used to control bleeding anywhere else in the gastrointestinal tract.

U.S. Pat. No. 6,952,615 B2 describes a cardiac balloon ablation catheter that has internal electrodes for heating the fluid and transmitting it to surrounding tissue. It has a sharp point that prevents it from being used in the gastrointestinal tract. Moreover, the size and shape preclude application in the stomach, duodenum, esophagus or colon.

U.S. Pat. No. 6,123,718 by Tu et al is another device useful for cardiac ablation but cannot be applied to the gastrointestinal tract due to the longitudinal configuration and the traumatic distal end.

Patent application Ser. No. 10/768,037 by Rioux et al is for a percutaneously introduced monopolar ablation balloon used to ablate tumor in residual cavities following sugery. Its size and shape and monopolar configuration preclude its use for hemostatic control of bleeding through endoscopic instruments.

Although, the prior art electrosurgical devices are useful, they often do not provide satisfactory operation for a number of reasons as outlined above.

3. Objects and Advantages

It is the object and advantage of this invention to provide for a multipolar cautery device for thermal treatment of tissues that is larger in diameter than the working channel of an endoscope, colonoscope or other specialized means of viewing the internal organs through natural orifices.

It is an object and advantage of this invention to allow multipolar cautery to be applied with excellent tissue apposition with tangential application to the concave internal portions of the body where thermal treatment of tissues is needed.

It is an object and advantage of this invention to allow multipolar cautery to be applied with excellent tissue apposition with en-face applications.

It is an object and advantage of this invention to allow multipolar cautery to be applied omnidirectionally with excellent tissue apposition with en-face, off-set, downstream or tangential applications.

It is an object and advantage of this invention to allow a larger diameter (>2 mm) multipolar endoscopic cautery device to maximize the surface area for tissue contact to reduce the time required to treat large lesions.

It is an object and advantage of this invention to provide for a bipolar device that can be used with the common widely available bipolar control units that are commercially available including but not limited to the ERBE, Conmed, Endostat and Valleylab

It is an object and advantage of this invention to provide for a rigid multipolar cautery probe that allows adequate application of tamponade pressure to cauterize bleeding blood vessels by coapting or welding the vessel walls together.

It is an object and advantage of this invention to provide for a thermal cauterizing device that does not suffer from the problem of sticking to the tissues during repeated thermal treatment as occurs with heater probe type devices.

It is an object an advantage of this device to provide for an endoscopically introducible convex multipolar cautery surface to allow close apposition to the concave luminal surfaces of the internal viscera and organs.

Further objects and advantages of my invention will become apparent from a consideration of he drawings and ensuing description.

### SUMMARY

In one aspect, the invention features an endoscopically introducible, bipolar cautery probe for engagement with and treatment of body tissue on the basis of tissue conduction of RF energy and subsequent thermal effect. The probe is sized and constructed for insertion into the body of a patient through a channel of an endoscope, colonoscope, cystoscope or bronchoscope. The probe includes a catheter shaft that defines a liquid filling lumen. An expandable liquid filled inflatable chamber at the distal end of the catheter shaft is in liquid receiving relationship with the liquid filling lumen. The catheter, with the chamber-defining wall in collapsed condition, is sized to pass through the channel of the endoscope. The chamber has an inflated diameter that is greater than the diameter of the transverse cross-section of the endoscope channel. The chamber has an inflated shape that is substantially spherical.

The expandable chamber is covered with two sets of electrical conductors of opposing polarity. The electrodes of different polarity are selectively sized and generally uniformly distributed in spaced apart pairs of opposite polarity, over the expandable chamber. The ratio of the width of the electrodes to the spacing between them is selected so as to provide, a predetermined minimum number of spaced apart pairs of electrodes and to allow omnidirectional multipolar treatment of tissue when the probe is projected from the distal end of the endoscope. The term multipolar, as used herein, means the electrosurgical use of a plurality of conductors which are arranged in fixed relationship with each other on a probe body for at least a bipolar contact with a precise treatment of tissue targets over a wide range of orientations of the device relative to the tissue target. These conductors may be spirally arranged, axially arranged, transversely arranged or may be point conductors over the entire surface of the expandable chamber. The electrical conductors may be metal, conductive paint or conductive polymer.

The probe, once the chamber is collapsed, can be inserted into the body through the working channel of the endoscope and thereafter the chamber can be inflated with liquid to create a rigid chamber and probe. RF current is passed through the conductors of opposing polarity on the now inflated expandable chamber. Then the chamber can be pressed against tissue, the tissue than shorts the conductors of opposite polarity with which it is in contact and this resistive transmission of RF energy through the tissue in contact with the conductors results in a coagulative ablative thermal effect. It can be used to treat a relatively large area of the tissue, because the inflated chamber is large compare to the diameter of the accessory channel of the endoscope.

In preferred embodiments, the inflatable chamber is made of non-elastic expandable plastic polymer that when distended with liquid usually water or saline or even compressed air is rigid and non-flexible. The device includes a plurality of spaced flexible electrical conductors on its chamber surface arranged spirally, axially, transversely or as discrete discs on the surface of the expandable chamber. The electrical conductors may be flexible metal strips, metal discs or conductive paint or conductive polymer. An external RF source is connected via two conductors that run the length of the shaft of the device to terminate in the electrical conductors on the surface of the expandable chamber. The chamber may be a foldable, expandable, substantially nonelastomeric balloon made of flexible plastic material. The material should have heat tolerance to at least 100 degrees centigrade as this is the temperature required for tissue cautery effect.

In one embodiment, the chamber is an expandable chamber that surrounds the distal end of the catheter shaft, and that extends axially at or just beyond the distal end of the catheter shaft when the balloon is inflated. In another embodiment, the chamber is disposed annularly around the distal portion of the catheter shaft in a manner such that, when filled with the liquid, the chamber extends distally beyond the end of the catheter shaft. The catheter shaft defines a lumen that extending through the catheter shaft and terminates at an opening in a distal end of the catheter shaft. The lumen may be used to irrigate the tissues with water during the procedure.

In another aspect, the invention features a method of treating tissue inside a patient's body. A user inserts an endoscope into the patient's body. A channel passes through the endoscope and terminates at an opening in a distal end of the endoscope. The user views the inside of the patient's body through the endoscope, to determine the location of tissue to be treated, and inserts a catheter shaft into the channel, in a manner such that a portion of the catheter shaft extends beyond the opening in the distal end of the endoscope. A expandable chamber, defined by a flexible wall, is mounted on the portion of the catheter shaft that extends beyond the opening in the distal end of the endoscope. The user fills the chamber with fluid, to cause the chamber to expand to a diameter greater than the transverse cross-section of the endoscope channel, and become rigid and positions the chamber at the location of tissue to be treated. The user may direct the chamber in an axial en-face direction or a tangential direction to the location of the tissue to be treated, and may apply pressure to the tissue as the tissue is heated, so that the tissue is compressed, thereby maximizing the vascular hemostatic effect.

Hemostatic multipolar cautery probes according to the invention can apply heat tangentially as well as en face, and can conform to the shape of the surface to which heat is being applied, to compress the tissue evenly and provide uniform heat transfer. Since the area of contact between the chamber and the tissue that is treated is relatively large, it is not necessary to reposition the chamber at multiple locations to ensure that an entire lesion is treated. Even if the user positions the probe somewhat off-center with respect to the lesion, the probe can nevertheless cause coagulation of bleeding arteries in the range of several millimeters in diameter. Hemostatic probes according to the invention can be used with relatively small endoscopes, because the chamber at the tip of the hemostatic probe is expandable to a diameter greater than the diameter of the transverse cross-section of the endoscope working channel. However, when collapsed the diameter is small enough to pass through the narrow working channel of the specialized flexible instrument. Thus, it is not necessary to switch to a larger endoscope when it is discovered upon viewing through the endoscope that the lesion to be treated is larger than was expected.

With an electrosurgical device in accordance with the invention, a bleeding tissue area can be approached over a broad range of orientations, that is omni-directionally and yet treated with greater effectiveness and fewer probe applications. A more uniform coagulation is achieved with a more predictable zone of coagulation.

The use of a multiple number of pairs of electrodes of different conductors over the surface of the expandable chamber assures at least bipolar or multiple bipolar tissue contact when the probe body is applied to the bleeding tissues. A particularly effective probe body in accordance with the invention employs a pair of flexible spiral electrodes, that run circumferentially from one pole to the other around the peripheral surface of a sphere shaped probe body. Bipolar, tripolar or higher polar tissue contact can be made independent of the orientation of the probe body for effective treatment of tissue such as gastric bleeding ulcers or vascular malformations

### DRAWINGS: FIGURES

FIG. 1 is a drawing of an endoscope and an expandable chamber multipolar catheter according to the invention, the catheter passing through the channel in the endoscope in deflated condition

FIG. 1A is a cross-section of an expandable chamber multipolar catheter of FIG. 1 with the chamber in the deflated condition

FIG. 1B is a cross-section of the catheter of FIG. 1A with the wings of the deflated expandable chamber or balloon folded to pass through the working channel of the endoscope

FIG. 2 shows the catheter of FIG. 1 in an inflated condition, on passing through a channel in the endoscope.

FIG. 3 is a view of a transverse cross-section of the shaft of the catheter of FIG. 2

FIG. 4 shows the expandable chamber multipolar catheter of FIG. 1 in an inflated condition and being used to treat tissue that is en-face to the endoscopist.

FIG. 4A shows the expandable chamber multipolar catheter of FIG. 1 in an inflated condition and being used to treat tissue that is tangential in relation to the endoscope

FIG. 4B shows the expandable spherical chamber multipolar catheter of FIG. 1 in an inflated condition and being used to treat tissue facing away from the endoscopist.

FIG. 5 shows the preferred embodiment of an expandable tip multipolar catheter according to the invention with spiral paired polar electrodes in inflated condition

FIG. 6 is an end view of the expandable tip multipolar catheter of FIG. 5

FIG. 7 is a view of the catheter of FIG. 5 where the expandable part joins the catheter shaft

FIG. 8 shows one embodiment of an expandable tip multipolar catheter according to the invention with transverse circular electrodes in inflated condition

FIG. 9 shows one embodiment of an expandable tip multipolar catheter according to the invention with discrete disc like electrodes in inflated condition

FIG. 10 shows one embodiment of an expandable tip multipolar catheter according to the invention with longitudinal axial electrodes

FIG. 11 is a transverse cross-section of the an expandable tip multipolar catheter of FIG. 10

FIG. 12 shows one embodiment of an expandable tip multipolar catheter according to the invention with a cylindrical shaped cautery probe with spiral paired polar electrodes in inflated condition.

FIG. 13 shows one embodiment of an expandable catheter according to the invention with a spherical shaped catheter probe with a unipolar electrode and the return circuit completed by a return electrode of large surface area affixed to the patient skin.

### DRAWINGS: REFERENCE NUMERALS

100 Imaging sensor on tip of endoscope

102 Illumination source on tip of endoscope

104 Shaft of bipolar expandable chamber cautery probe

106 Expandable chamber or balloon

108 Shaft of the endoscope

110 Rigid member and water irrigation channel

112 Working accessory channel in endoscope

114 Wings formed by the deflated expandable chamber or balloon

116 Folded wings of deflated expandable chamber or balloon

300 Channel to fill expandable chamber

302 Electrical conductor (+) positive polarity

304 Electrical conductor (−) negative polarity

306 Water tissue irrigation channel

400 Concave tissue surface to be treated

1301 Unipolar electrode

1302 Conductor for unipolar electrode to external electrical source

1303 Large surface area return electrode

1304 Skin of body

D1 Diameter of deflated expandable catheter chamber

D2 Diameter of inflated expandable catheter chamber

### DETAILED DESCRIPTION

Structure

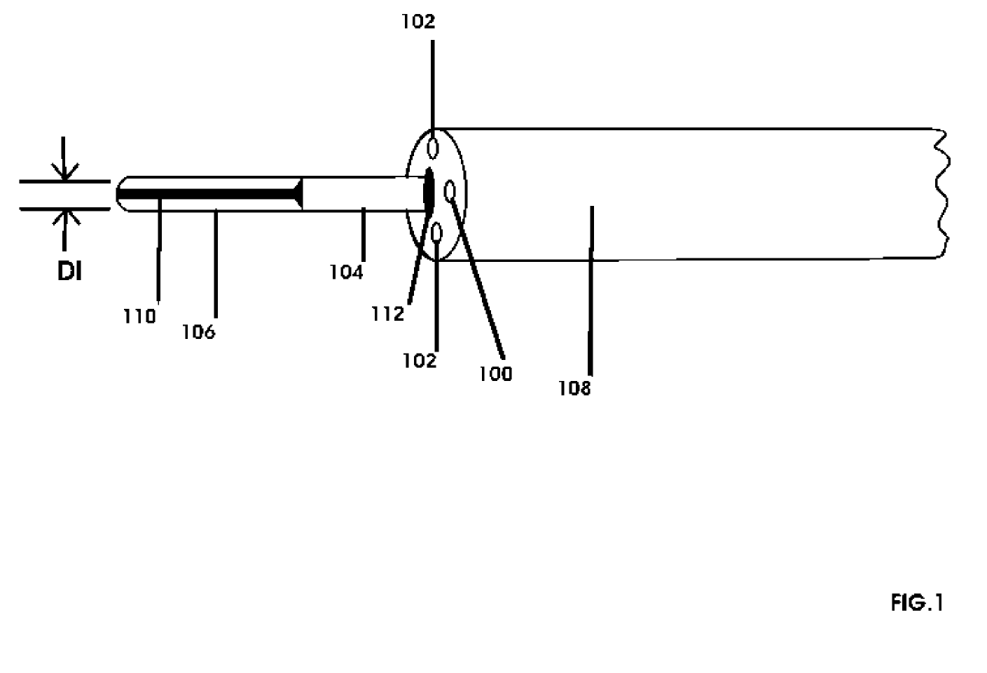

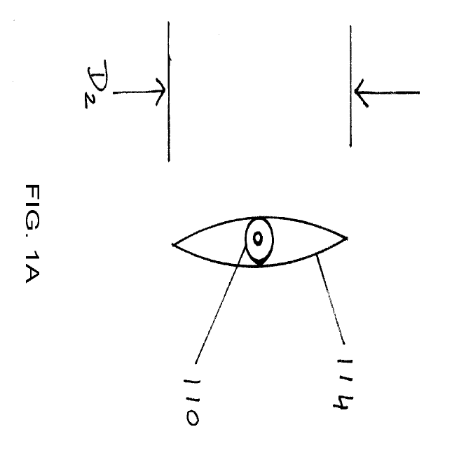

Referring to FIG. 1, an endoscope 108, is introduced into a cavity of the body through a natural duct or passageway and is cylindrical in shape. A pair of light sources 102 and an image sensor 100 are located on the distal end of the endoscope. The endoscope 108 is used for viewing the inside of the patient's body, to determine the location of a lesion such as an ulcer in the stomach, other gastrointestinal bleeding, bleeding in the colon or bleeding in the lung. A channel passes through the length of the endoscope and terminates in an opening 112 in the distal end of the endoscope. The channel diameter is typically 2.8 millimeters or smaller but can be as large as 3.2 millimeters or more.

The hemostatic balloon probe is formed of an electrically insulative catheter shaft 104 that at its distal end has an expandable balloon 106. The catheter shaft 104 is long, typically 180 centimeters to 300 centimeters to traverse the entire length of the endoscope 108 and project out of the channel 112 beyond the distal tip of the endoscope 108. The catheter shaft is made of engineering plastic. A hemostatic balloon probe 104 is insertable through the channel of endoscope 108 when the balloon 106 is deflated, as shown in FIG. 1. The catheter shaft has a diameter D1 of 5 French, 7 French, or 10 French to fit through the channel 112 of the endoscope 108. The balloon when collapsed has a diameter D1 substantially similar to the diameter of the catheter shaft 104. Through the center of the balloon runs an extension of the catheter shaft 110, that provides support for the balloon at its distal end and also provides for a tissue irrigation channel that terminates in a small opening at the distal end of the balloon for irrigating the tissues that needs to be treated.

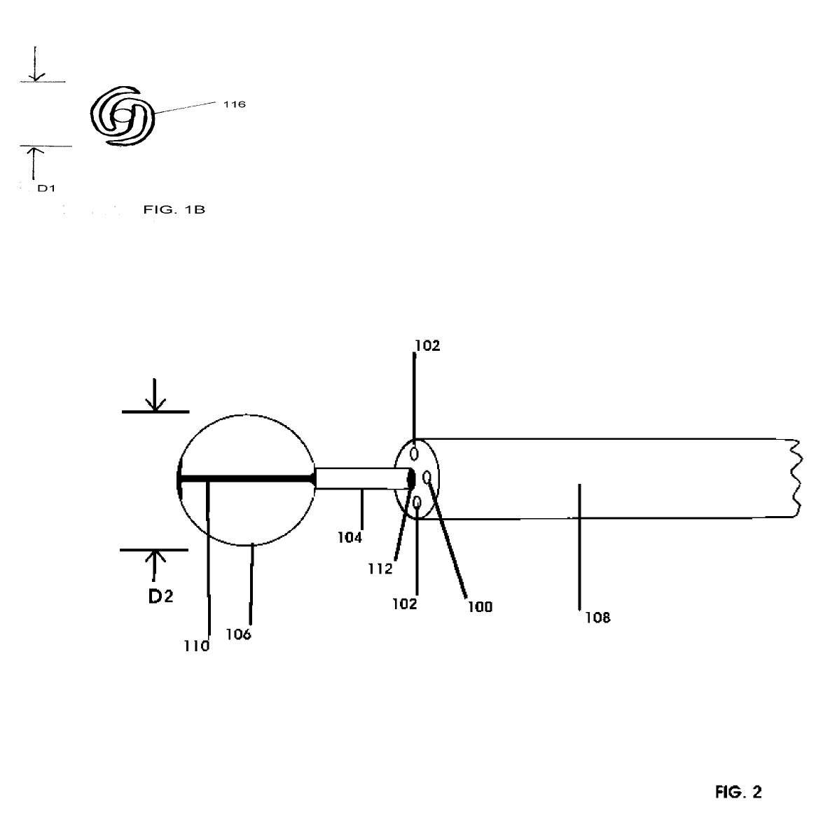

Referring to FIG. 1A, the balloon is shown collapsed with a winged appearance when viewed from the distal end, the supporting central extension of the catheter shaft 110 is also seen. In FIG. 1B, the collapsed balloon has been rolled or furled around the central shaft 110, to provide a size and shape for introduction through the channel 112 of the endoscope 108. After the probe has been passed through the channel 112 of the endoscope 108 and is projecting from the distal end of the endoscope the balloon can then be inflated to the shape of a sphere, as shown in FIG. 2. The balloon when fully inflated typically has a diameter D2 of approximately three to five millimeters. The ratio of inflated diameter D2 to deflated diameter D1 is variable depending on the particular application but ranges from 1.2 to 3.0.

In the embodiment shown in FIGS. 1 and 2, the hemostatic balloon probe includes the balloon 106 mounted on a plastic catheter shaft 104. The balloon 106 is radially expandable over the distal extension of the catheter shaft 110. The balloon 106 may be either an elastic polymer balloon or preferably a foldable, non-elastomeric balloon. If the balloon is elastic, it will conform to a lesion and distribute pressure evenly to the zone to be coagulated without leaving gaps between the balloon and the lesion. If the balloon is a foldable furling type non-elastomeric balloon it will be rigid when inflated and provide for excellent tamponade of the tissues which is very helpful when trying to control bleeding from leaking blood vessels in the gastrointestinal tract. The elastic balloon may be made of silicon rubber, which is flexible, does not stick, and can tolerate high temperatures of 100 degrees centigrade or more. The furling type noncompliant balloon may be made of engineering plastic that can tolerate high temperatures such as polytetrafluroethylene (PTFE) or perfluoroalkoxy fluorocarbon (PFA) or fluoroethylenepropylene (FEP) or polyethylene terephthalate (PET) etc. These engineering plastics can tolerate high temperatures and are flexible but non-elastomeric. The balloon 106 is fillable with any suitable fluid such as air or water or saline via an external syringe. The exterior of the balloon 106 may be coated with a non-stick coating having a low coefficient of friction, such as silicone, teflon or polysiloxane.

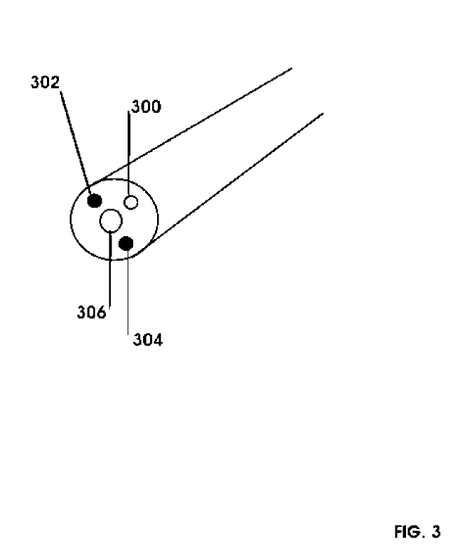

A cross-section of the catheter shaft 104 is shown in FIG. 3. The catheter shaft contains electrically insulated conductor 302 and 304 that allow the transmission of electrical potential from an external source such as the standard and widely used medical electrosurgical generators by ERBE or Valley Forge or Endostat. The conductors 302 and 304 run the length of the catheter shaft and terminate in the bipolar or multipolar electrodes on the expandable balloon 106. These conductors 302 and 304 are made of copper and are covered with plastic insulation. The conductors are connectable to the standard widely used radiofrequency electrosurgical generator via a standard 2 pin round bipolar connector cable. In endoscopic medical applications radiofrequency electrosugical generators are generally used to provide a wattage of typically 15-40 watts. Radiofrequency electrical potential is used in the medical field to prevent neuromuscular excitation and electrocution.

The catheter shaft also defines a fluid filled lumen 300 that runs the length of the catheter shaft and is in communication with the expandable balloon 106. The fluid filled lumen 300 allows the introduction and withdrawal of fluid (air, water or saline etc.) from the balloon 106 to alternatively expand or collapse the balloon as needed. A standard 2 ml or 5 ml syringe may be used to accomplish the introduction and withdrawal of fluid from the balloon 106 via fluid filled lumen 300. The connector between the syringe and the fluid filled lumen 300 is a standard medical Luer lock connector, it is the most widely used connector in the medical field and it is used for connecting conduit fluid transmitting tubing. The fluid filled lumen 306 is a channel or conduit that runs the length of the catheter shaft 104 and extends to the distal end of the balloon 106 through the distal extension of the catheter shaft 110 and allows for irrigation of tissue with fluid such as saline or water that is used to wash blood or debris away to enable unobstructed viewing of the lesion. The lumen 306 that runs through the length of the catheter shaft 104 may alternatively provide a conduit for a guidewire. The guidewire exits the catheter shaft 104 through an opening in the tip of the catheter shaft extension 110. The balloon 106 is annularly disposed around the catheter shaft extension 110 and expands radially.

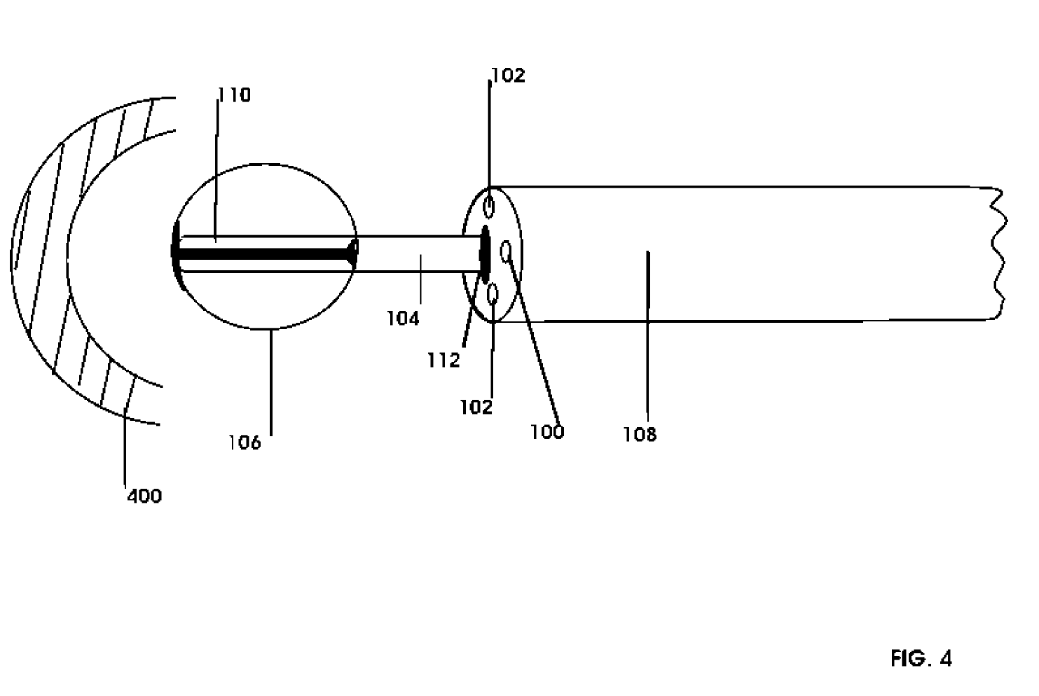

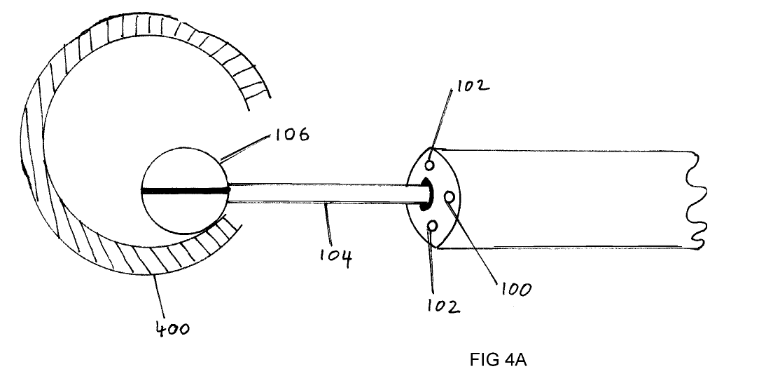

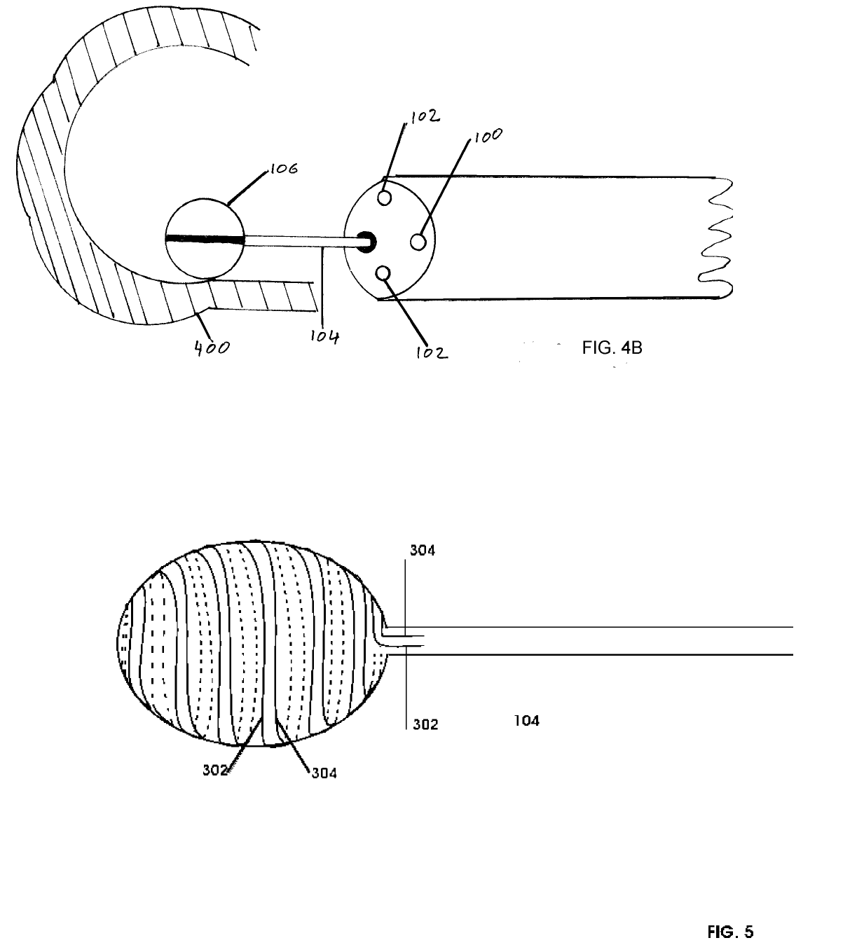

The expanded balloon 106 can be pressed against tissue en face to treat the tissue 400 and control bleeding or ablate lesions as shown in FIG. 4. In FIG. 4A the balloon 106 is pressed to treat the tissues 400 facing downstream and away from the tip of the endoscope. The balloon 106 is pressed to the tissues in tangential fashion as shown in FIG. 4B. The spherical configuration of the balloon 106 allows omni-directional treatment of the tissues. Moreover, the spherical configuration of the balloon allows for close fitting of the convex bipolar or multipolar treatment surface to the tissues in the body cavity which predominantly define a concave configuration in the esophagus, stomach, duodenum, jejenum and colon.

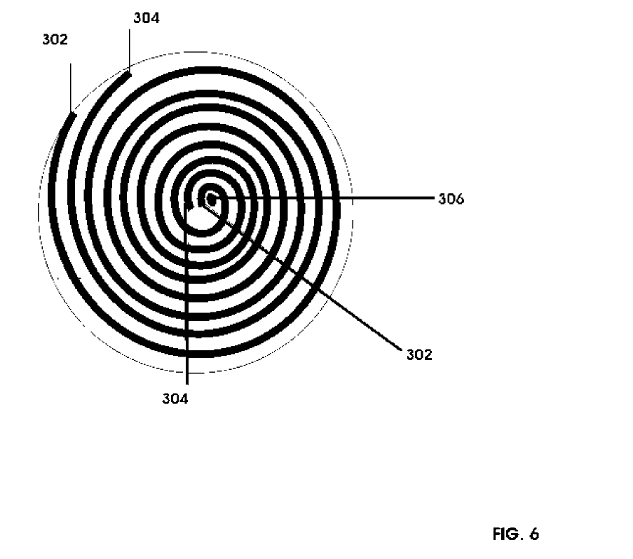

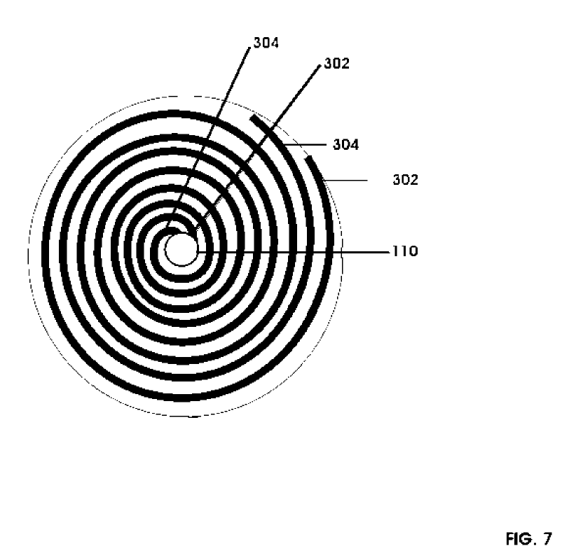

Referring to FIG. 5, the preferred embodiment of the device is shown with a balloon at the distal end of the catheter shaft 104 covered with circumferential, parallel and spirally disposed bipolar electrodes 302 and 304. Proximally electrodes 302 and 304 are in continuity with the conductors 302 and 304 within the shaft of the catheter 104. From the proximal end of the balloon FIG. 7 the electrodes 302 and 304 run a parallel course encircling the balloon in a spiral fashion to terminate at the distal most tip of the balloon as shown in FIG. 6. One embodiment of the termination of the electrodes 302 and 304 in relation to the opening of the irrigation channel 306 is shown in FIG. 6. The width of the electrodes and the space between the electrodes is optimized to provide effective tissue resistive conduction, and will vary depending on the particular application and in particular the size of the balloon. The electrodes 302 and 304 on the balloon are made of thin flexible metallic strips to allow folding or furling of the balloon during insertion. Suitable metals for the electrodes on the balloon include nitinol, gold, silver or copper. Alternatively the electrodes may be painted on the balloon and made of conductive paint or may be made of conductive polymer bonded to the surface of the balloon such as polyaniline. Compounds and techniques for manufacture for this purpose are well known in the electronic and medical manufacturing process. In one embodiment of the invention the ratio of the width of the electrodes to the space between the electrodes is 1:2 to 2:1. Typical cross-section diameter dimension of the device with the balloon collapsed may be 2.2 mm to allow introduction through the channel of a diagnostic upper endoscope, on inflation the balloon may expand to a diameter size of 3.5 mm. The electrodes 302 and 304 may each have a width of 1.0 mm and gap or space between the electrodes of 0.7 mm.

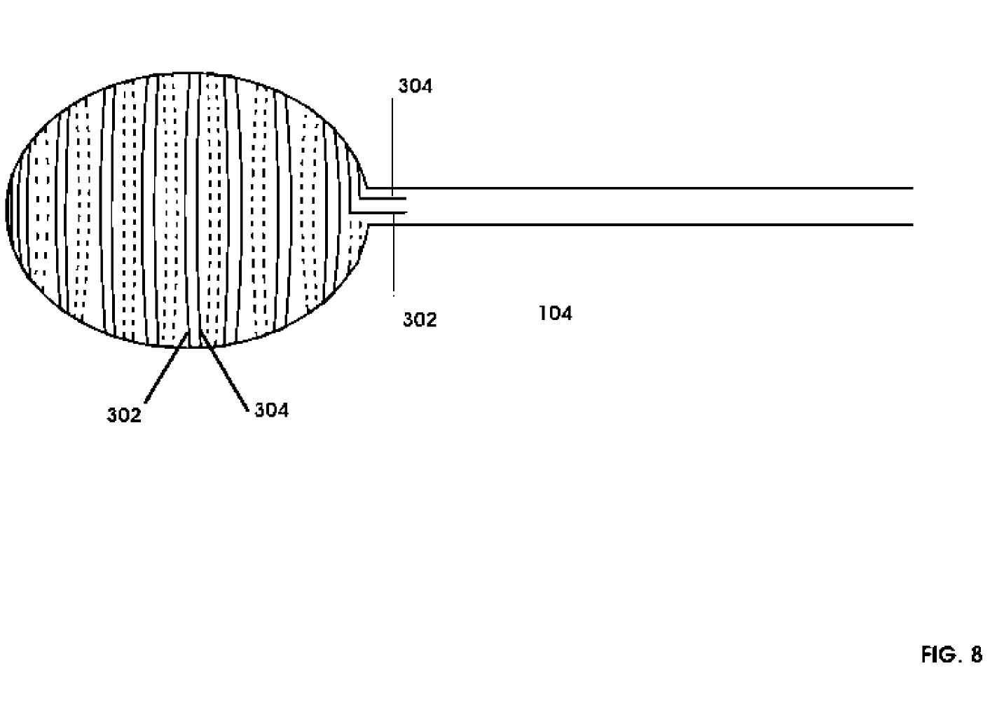

An alternative embodiment of the invention is shown in FIG. 8. The electrodes 302 and 304 are uniformly disposed in a transverse, circumferential and parallel arrangement around the balloon 106 to provide a plurality of multipolar treatment surface on the expandable balloon 106. In one embodiment of the invention the ratio of the width of the electrodes to the space between the electrodes is unity.

Another embodiment of the invention is shown in FIG. 8. The electrodes 302 and 304 are disposed as uniformly distributed discrete round discs on the surface of the balloon to provide a multipolar treatment surface on the expandable balloon 106. The discs are disposed in an alternating polarity arrangement to provide at least bipolar or higher polar treatment effect.

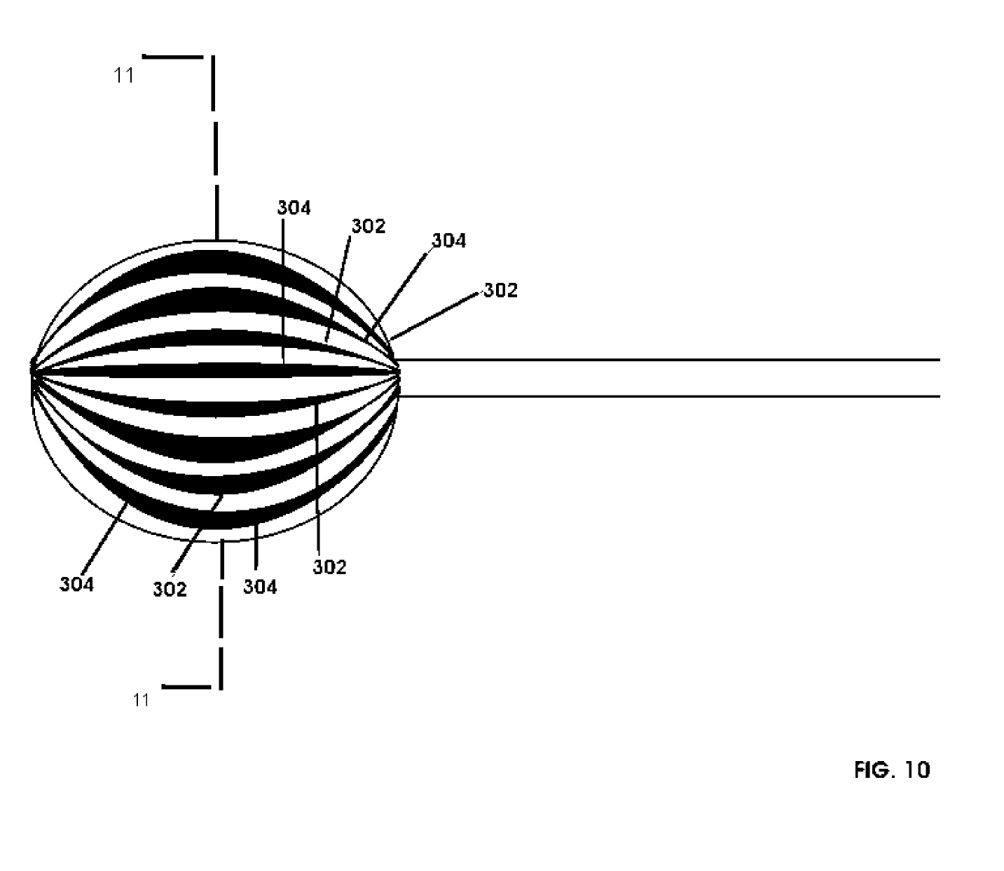

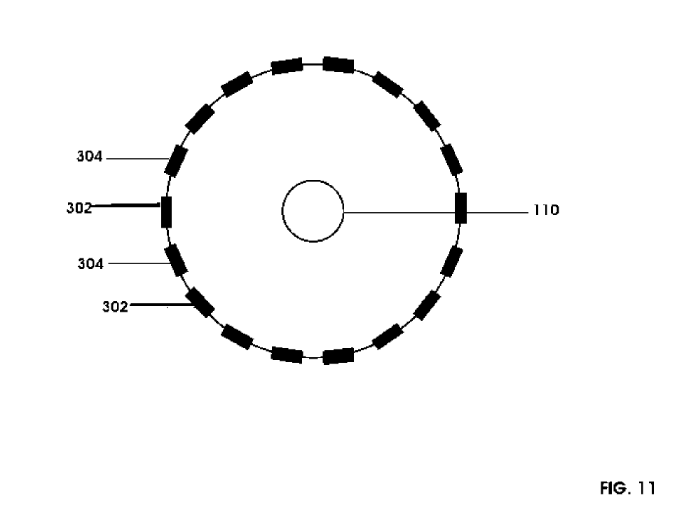

An alternative embodiment of the invention is shown in FIG. 10. The balloon 106 has an oval configuration. The electrodes 302 and 304 are uniformly disposed longitudinally in alternating arrangement on the balloon. The electrodes are of alternating positive and negative polarity to provide at least a bipolar treatment surface. The width of the electrodes 302 and 304 changes from narrow at the poles of the balloon to wide in the middle portion to allow for a constant ratio of the width of the electrode to the space between the electrodes. In one embodiment of the invention the ratio of the width of the electrodes to the space between the electrodes is unity. FIG. 11 is a transverse cross section view of the expandable balloon cautery probe shown in FIG. 10. The electrodes 302 and 304 are disposed in alternating arrangement. The catheter extension shaft 110 is shown running through the middle of the balloon, it provides for a tissue irrigation channel and provides an attachment point at the distal end of the balloon.

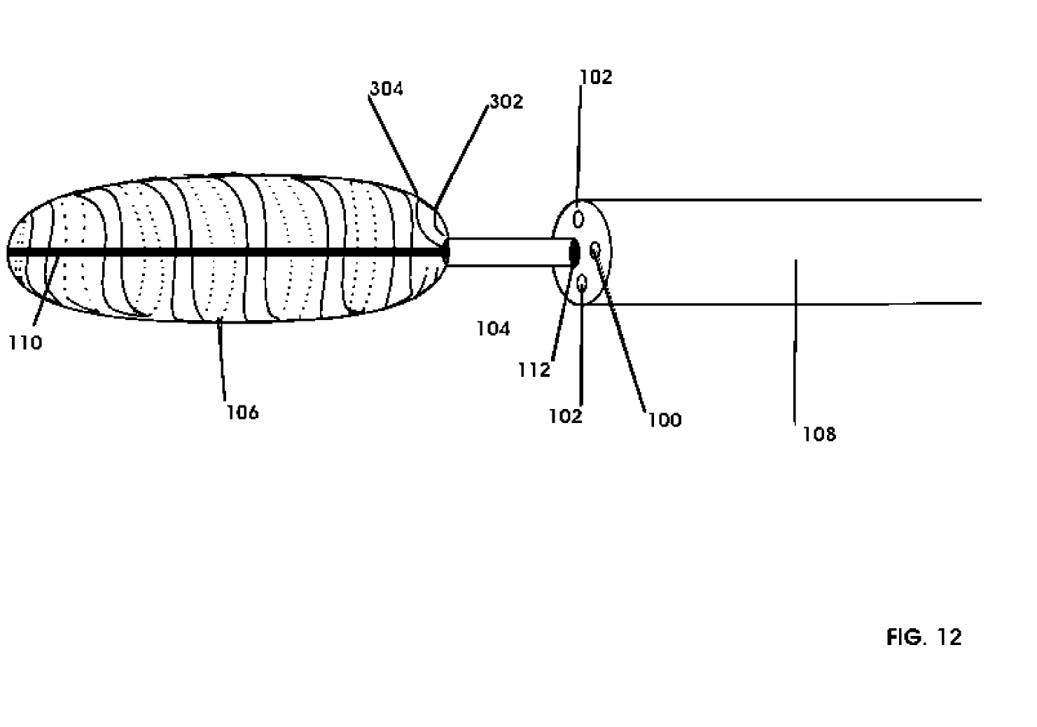

Another embodiment of the invention is shown in FIG. 12. The balloon 106 has a cylindrically shape with a blunt convex distal end. In this embodiment of the device the cylindrical balloon 106 at the distal end of the catheter shaft 104 is covered with circumferential, parallel and spirally disposed bipolar electrodes 302 and 304. In one embodiment of the invention the ratio of the width of the electrodes to the space between the electrodes may range from 1:2 to 2:1. Typical cross-section diameter dimension of the device with the balloon collapsed may be 2.2 mm to allow introduction through the channel of a diagnostic upper endoscope, on inflation the balloon may expand to a diameter size of 3.5 mm. The length of the balloon 106 may be of the order of 7.6 mm. The electrodes 302 and 304 may each have a width of 1.0 mm and gap or space between the electrodes of 0.7 mm.

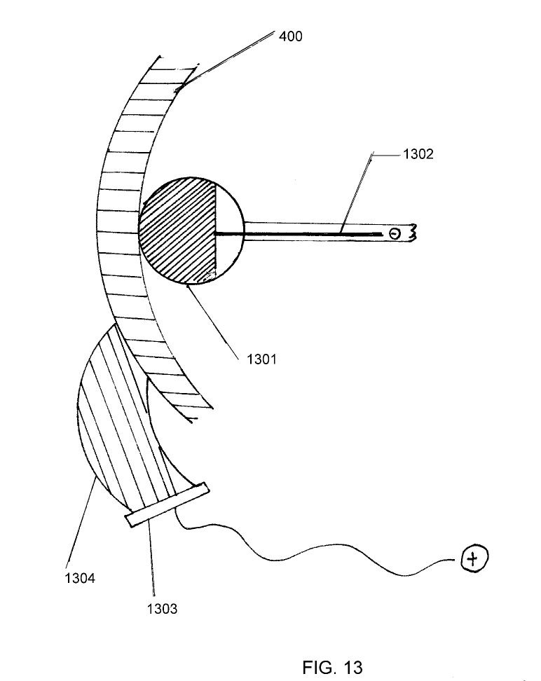

An alternative embodiment of the invention is shown in FIG. 13. The expandable endoscopically introducible balloon probe 1301 has a monopolar configuration. A single conductor 1302 extends the length of the catheter shaft and terminates in a monopolar electrode that covers a substantial distal portion of the expandable chamber. The electrical circuit is completed by a patient grounding pad of large surface area 1303 placed on the skin of the patient 1304. The small contact area of the balloon 1301 with the tissue to be treated 400 compared to the large surface area between the skin 1304 and the return grounding pad electrode 1303 results in high resistance to electrical transmission at the treatment site compared to the rest of the circuit and hence a thermal electrocautery treatment effect where the balloon 1301 makes contact with the tissue 400 to be treated. Radiofrequency electrical energy ranging from 15-30 watts may be applied for 2-30 seconds to provide a treatment effect.

### Operation

Referring to FIG. 1, endoscope 108 is insertable through a natural orifice and duct into a patient's body. Once the endoscope 108 has been inserted into the patient's body, it is used for viewing the inside of internal organs such as the stomach, other parts of the gastrointestinal system, the lung, etc., to determine the location of a bleeding lesion. The hemostatic probe 104, with the balloon 106 in its deflated state, is inserted through the channel 112 that passes through the length of the endoscope. The balloon 104 is positioned beyond the distal end of the endoscope. Balloon 104 is inflated through lumen 300 with either saline, water or air. The balloon is placed en face against the lesion to be treated, and is pressed against the lesion. The user then selects the wattage of the radiofrequency electrical potential to be applied to the electrodes from the standard electrosurgical generator such as ERBE or Valley Forge. For control of bleeding blood vessels from ulcers in the stomach or duodenum a typical setting of 15-30 watts is used. For ablation of arteriovenous malformations in the colon a setting of 10-20 watts may be typically used. The transmission of the electrical potential through the local tissues in contact with the bipolar or higher polar electrodes results in tissue resistive conduction to complete the circuit, which in turn leads to the generation of thermal energy that results in a coagulative ablative effect. The combination of mechanical pressure and thermal coagulation results in coaptation or welding together of the walls of bleeding blood vessels and hence control of bleeding. The combination of heat and pressure causes coagulation of the lesion.

The tissue coagulation zone is not limited to the size of the hemostatic probe or the diameter D1 of the endoscope channel. Because the balloon expands to a diameter D2 greater than the diameter of the transverse cross-section of the endoscope channel 112, it is not necessary to preselect an endoscope having a large diameter, or to switch to a larger endoscope when it is discovered that the tissue zone to be treated is large. If it is not practicable to place the balloon en face against the tissue to be treated, because of the spherical shape of the balloon adequate omni-directional tissue treatment effect can be obtained with tangential application of the balloon or downstream application of the balloon.

With a multipolar device in accordance with the invention, electrocoagulation can be obtained with various orientations of the probe body relative to the tissue and without requiring a rotation of the probe body. This is particularly advantageous when the device is used through an endoscope so that end-on, oblique, tangential or sidewise applications of the probe results in at least a bipolar contact.

With a multipolar device in accordance with the invention, the electric field pattern around the probe body may be selected to provide homogeneous thermal heating close to the tissue surface in contact with the probe body. For example, in the above description of the device, the radial extent of the electrical field is a function of the size of the gap between conductor electrodes. Thus, for some applications where a lesser radial electrical field and depth of injury is desired to reduce the depth of coagulation, the gap between the electrodes 302 and 304 may be reduced. In such case a larger number of electrodes can be employed resulting in a greater number of bipolar contacts. When a deeper tissue treatment is needed, the gap or space between electrodes may be increased. The width of conductors and gap sizes may thus be selected, depending upon the particular tissue being treated.

Some of the considerations in the selection of the width of electrode (W) to spacing of electrode (S) ratio relate to the heat distribution achieved in the tissue to be treated and the generation of tissue sticking problems. For example, a tissue sticking problem arises when a high concentration of heat causes too high a temperature in the tissue, generally greater than about 200. degree. F., thus resulting in the adherence of tissue to metal parts of the probe body. If such condition occurs, the probe body requires frequent removal for cleaning and undesirably extends the duration of the treatment of the patient. When such excessive amount of heat is applied to stop a bleeding area, the resulting sticking of cauterized tissue also makes it difficult to disengage the probe body without removing the coagulated layer and thus restarting bleeding.

Preferably, just enough electrical power, generally in the range from about 10 watts to about 30 watts for a 2.3 mm diameter probe, should be applied to thermally coagulate the tissue area in contact with the probe to stop bleeding. The electrical power further should be applied in such manner that high voltage punch-through of cauterized dried tissue leading to sticking and/or unnecessary tissue wall damage is avoided. The electrical power normally is supplied in pulses having a duration of the order of one or several seconds.

Tissue sticking problems can be substantially avoided with a multipolar device in accordance with this invention because it enables the application of an adequate amount of electrical power at a relatively low voltage. The amount of power that can be applied is a function of the surface area of the probe electrodes 302 and 304 brought into contact with the tissue. When the surface area is relatively large, i.e. with an adequate conductor or electrode width, W, to spacing, S, ratio, there exists sufficient surface contact between an electrode and the tissue to supply electrical power at a relatively safe low voltage which is unlikely to force power through a dessicated layer causing deeper damage and risk of perforation.

The electrode to tissue contact area tends to be a function of the ratio of the conductor width, W, to the spacing, S between conductors. At a low ratio, say less than about 1:3 or expressed in a fraction ⅓, the minimum amount of power needed to stop bleeding requires a voltage that is likely to be above the safe operating range. At such lower W:S ratio of about ⅓ the multipolar probe may provide the desired coagulating function; however, the impedance or resistance between the probe and tissue with such low ratio tends to be higher because the conductor surface in contact with tissue is less, thus requiring a higher voltage to transfer the desired amount of power into the tissue. This higher voltage tends to result in less uniform heating with hot spots that are likely to cause tissue sticking.

The W:S ratio, of the conductor width, W, to spacing, S, thus should be greater than about one-third (⅓) below which value less uniform heating with likelihood of sticking tends to occur. Preferably the W:S ratio is not less than about one-half (½). At W:S ratios of about 1:1 and 2:1 the probe tends to function adequately with good uniform heating. With a W:S ratio of 3:1, or expressed as 3, there is a tendency for less uniform heating but the presence of a relatively larger conductor surface area enables operation at a lower voltage which is safer from a standpoint of avoiding tissue sticking. Generally W:S ratios ranging from 1:1 to 1:2 is preffered.

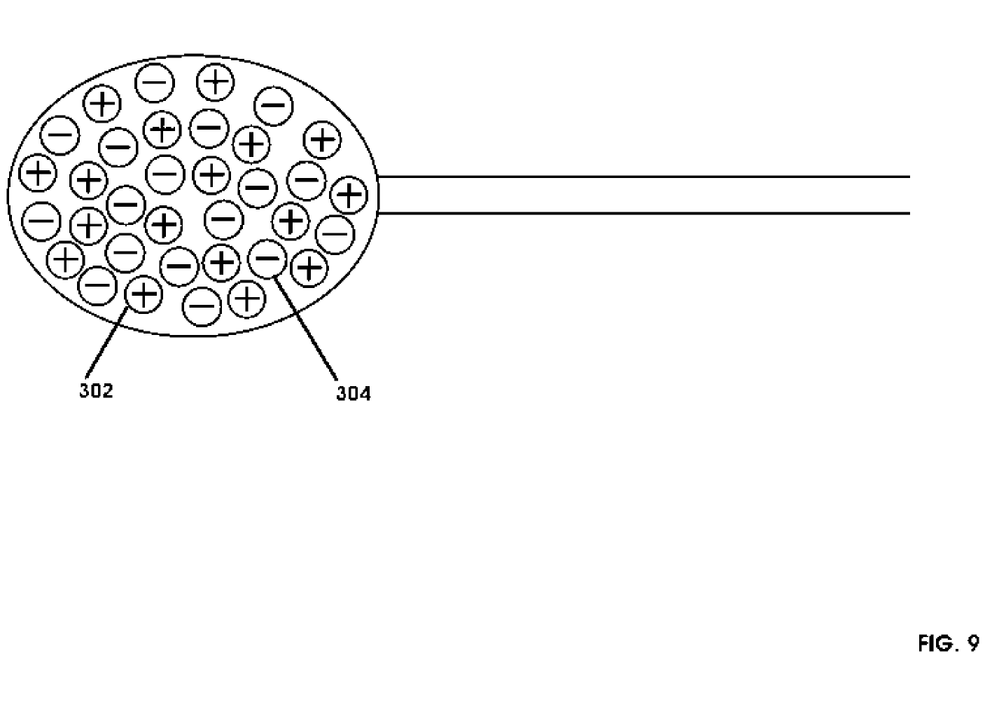

With the geometrical arrangement and distribution of electrodes on bipolar or multipolar device as shown in FIGS. 4-12, the advantages of bipolar or multipolar tissue treatment are obtained and, in particular, an ability to randomly approach a tissue target area either side-wise, head-on, tangentially or obliquely, without a loss of an ability to treat the target area. The incorporation of a central wash channel further enhances the utility of the device. Because the balloon expands to a diameter greater than the diameter of the transverse cross-section of the endoscope channel, it is not necessary to preselect an endoscope having a large diameter, or to switch to a larger endoscope when it is discovered that the tissue zone or bleeding vessel to be treated is large.

Variations from the described embodiments may be made by one skilled in the art without departing from the scope of the invention.

What is claimed is:

1. An endoscopically introducible, multipolar probe, for engagement with and treatment of body tissue on the basis of tissue resistive conduction, said probe being sized and constructed for insertion into the body of a patient through a working channel of an endoscope, said channel having a transverse cross-section of a predetermined diameter, said probe comprising
a catheter shaft defining a fluid filling lumen,
means at the distal end of said catheter shaft defining a collapsible fluid expandable chamber in fluid receiving relationship with said filling lumen,
said catheter, with the chamber-defining wall in collapsed condition, being sized to pass through said predetermined channel of said endoscope,
said chamber having an inflated diameter that is greater than the diameter of the transverse cross-section of said endoscope channel,
said collapsible fluid expandable chamber covered with a plurality of electrodes in spaced apart relationship,
said plurality of electrodes connectable via means to an external radio frequency electrical energy source,
whereby said probe, when said chamber is deflated, can be inserted into said body through said endoscope and thereafter said chamber can be inflated with fluid to create an enlarged surface area, and said radio frequency electrical potential from said external power source applied to said electrodes on surface of said inflated chamber and said chamber extending beyond the end of said endoscope can be pressed against tissue to press said multipolar electrodes to said tissue to treat by local tissue resistive bipolar or multipolar cautery a larger area of tissue relative to the size of said working channel of said endoscope.

2. The endoscopically introducible probe of claim one wherein said wall of said collapsible fluid inflatable chamber comprises a foldable substantially non-elastomeric balloon.

3. The endoscopically introducible probe of claim one wherein said wall of said collapsible fluid inflatable chamber comprises an elastomeric balloon.

4. The endoscopically introducible probe of claim 1 wherein said walls of said chamber comprises a balloon that surrounds the distal end of said catheter shaft.

5. The endoscopically introducible probe of claim 1 wherein said catheter shaft defines a second lumen extending through said catheter shaft and terminating at an opening in distal end of said expandable balloon to provide means for tissue irrigation or passage of a guidewire.

6. The endoscopically introducible probe of claim 1 wherein said expandable chamber comprises a convex distal portion covered with said electrodes to provide multipolar or bipolar contacts around the distal end of the probe body.

7. The endoscopically introducible probe of claim 1 wherein said electrodes comprise metal strips fused to the surface of said expandable chamber.

8. The endoscopically introducible probe of claim 1 wherein said electrodes comprise conductive paint or conductive polymer fused to the surface of said expandable chamber.

9. The endoscopically introducible probe of claim 1 wherein said expandable chamber comprises a substantially spherical shape with at least the distal end covered with said electrodes.

10. The endoscopically introducible probe of claim 1 wherein said expandable chamber comprises a substantially generally smooth external surface.

11. The endoscopically introducible probe of claim 1 wherein said expandable chamber comprises a substantially cylindrical shape with a blunt distal end that is covered with said electrodes.

12. The endoscopically introducible probe of claim 1 wherein said probe has a plurality of electrodes that cover the surface of said expandable chamber.

13. The endoscopically introducible probe of claim 1 wherein said electrodes being so selected and positioned as to enable effective bipolar or multipolar treatment of tissue with effectively omnidirectional probe body orientations relative to the tissue to be treated.

14. The endoscopically introducible probe of claim 1 wherein said electrodes being so selected and positioned to align to the longitudinal axis of said probe along the peripheral surface of said expandable chamber to provide bipolar or multipolar treatment of tissue.

15. The endoscopically introducible probe of claim 1 wherein said electrodes are formed with circular bands located on surface of said expandable chamber and extending along the longitudinal axis of the probe to provide bipolar or multipolar treatment of tissue.

16. The endoscopically introducible probe of claim 1 wherein said electrodes comprise a pair of bipolar electrodes being so selected and positioned in a spiral configuration on the surface of said expandable chamber extending substantially from the proximal to distal end of said expandable chamber to provide omni-directional bipolar or multipolar treatment of tissue by local tissue resistive conduction.

17. The endoscopically introducible probe of claim 1 wherein said electrodes comprise a plurality of disc shaped electrodes being so selected and positioned on surface of said expandable chamber to provide bipolar or multipolar treatment of tissue by local resistive tissue conduction.

18. The endoscopically introducible probe of claim 1 wherein said electrodes comprise a plurality of interposed electrodes of opposite polarity being so selected and positioned on surface of said expandable chamber to provide bipolar or multipolar treatment of tissue.

19. The endoscopically introducible probe of claim 1 wherein said expandable chamber consists of an insulative material and electrically isolated electrodes mounted on surface of said expandable chamber, include means to connect said electrodes to an external source of electrical energy, said electrodes of opposite polarity being respectively interposed with each other in fixed relationship on the peripheral surface of said expandable chamber, said electrodes of opposite polarity being further respectively so sized and distributed so as to extend in spaced apart pairs over the surface of said inflated expandable chamber and being arranged on the surface of said inflated expandable chamber so as to enable at least bipolar treatment of tissue with effectively omnidirectional orientations of the probe body relative to the tissue to be treated.

20. The endoscopically introducible probe of claim 1 wherein the application of said electrical power to the tissue to be treated through said spaced apart electrodes which are so sized and located that the ratio of the width of the conductors to the spacing between them is sufficient to obtain uniform heating and coagulation without sticking to the tissue.

21. The endoscopically introducible probe of claim 1 wherein the distal end of said catheter shaft and said expandable chamber in inflated condition comprise a substantially rigid structure to provide for application of substantial tamponade pressure on the tissue without flexure of said chamber or said catheter shaft.

22. A method of treating tissue inside a patient's body, comprising the steps of inserting an endoscope into a patient's body, said endoscope having a working channel that passes through said endoscope and that terminates at an opening in a distal end of said endoscope, said channel having a transverse cross-section of a predetermined diameter, viewing the inside of the patient's body through said endoscope, to determine the location of tissue to be treated, positioning a catheter shaft within said channel in a manner such that a portion of said catheter shaft extends beyond said opening in the distal end of said endoscope, an expandable chamber being mounted on said portion of said catheter shaft that extends beyond said opening in the distal end of said endoscope, said chamber being defined by a flexible wall, and said chamber having a plurality of electrodes on its surface filling said chamber with fluid, said chamber when filled with said fluid having a diameter greater than the diameter of the transverse cross-section of said endoscope channel, positioning said chamber in contact with said tissue to be treated, and applying pressure on said tissue with said inflated chamber to bring said electrodes into contact with said tissue by local tissue resistive bipolar or multipolar cautery treating a larger area of said tissue relative to the size of said working channel of said endoscope.

23. The method of claim 22, wherein the step of positioning said chamber at the location of tissue to be treated comprises directing said chamber in a longitudinal axial direction to the location of the tissue to be treated.

24. The method of claim 22, wherein the step of positioning said chamber at the location of tissue to be treated comprises directing said chamber in a tangential direction to the location of the tissue to be treated.

25. The method of claim 22, wherein the step of positioning said chamber at the location of tissue to be treated comprises directing said chamber in an en-face direction to the location of the tissue to be treated.

26. An endoscopically introducible probe for treatment of the tissues of the body by tissue resistive conduction comprising
a catheter shaft, sized to pass through the working channel of an endoscope, said working channel having a transverse cross-section of predetermined diameter,
a collapsible and expandable chamber at the distal end of said catheter shaft, said chamber when collapsed having a diameter small enough to pass through the channel of said endoscope, said chamber when expanded having a diameter greater than said channel of said endoscope,
said chamber covered on its surface with one or more electrodes connectable via means to a source of electrical energy potential,
whereby said probe when said chamber is collapsed, can be inserted through said channel in said endoscope to the area of body to be treated, once said chamber is beyond the distal end of said endoscope, said chamber is expanded, an electrical potential is applied to said electrodes, and said chamber is then pressed on tissue to be treated, to thereby press said electrodes on tissue and by resistive tissue conduction treating a larger area of said tissue relative to the size of said channel of said endoscope.

27. An endoscopically introducible probe of claim 26 wherein said expandable collapsible chamber has a substantially spherical shape.

28. An endoscopically introducible probe of claim 26 wherein said expandable collapsible chamber has a substantially cylindrical shape with a blunt distal end.

29. An endoscopically introducible probe of claim 26 wherein said chamber is covered with a single unipolar electrode for tissue resistive treatment, with the electrical circuit completed through a large surface area return electrode affixed to the skin of the patients body.

30. An endoscopically introducible probe of claim 26 wherein said chamber is covered with at least one pair of electrodes of opposite polarity in spaced apart relationship for bipolar or multipolar treatment of tissue by local tissue resistive conduction.
